(19) United States
(12) Patent Application Publication
(10) Pub. No.: US 2013/0040939 A1
(43) Pub. Date: Feb. 14, 2013

(54) Oral Suspension Formulations of Esclicarbazepine Acetate

(71) Applicants: Teófilo Cardoso de Vasconcelos, S. Mamede do Coronado (PT); Ricardo Jorge dos Santos Lima, S. Mamede do Coronado (PT); Rui Cerdeira de Campos Costa, S. Mamede do Coronado (PT); Pedro Miguel da Costa Barrocas, S. Mamede do Coronado (PT); Ligia Sofia de Castro Pereira, S. Mamede do Coronado (PT)

(72) Inventors: Teófilo Cardoso de Vasconcelos, S. Mamede do Coronado (PT); Ricardo Jorge dos Santos Lima, S. Mamede do Coronado (PT); Rui Cerdeira de Campos Costa, S. Mamede do Coronado (PT); Pedro Miguel da Costa Barrocas, S. Mamede do Coronado (PT); Ligia Sofia de Castro Pereira, S. Mamede do Coronado (PT)

(73) Assignee: BIAL - PORTELA & CA, S.A., S. Mamede do Coronado (PT)

(21) Appl. No.: 13/394,414

(22) PCT Filed: Sep. 10, 2010
(86) PCT No.: PCT/PT10/00038

§ 371 Date: Oct. 25, 2012

Related U.S. Application Data
(60) Provisional application No. 61/241,195, filed on Sep. 10, 2009.

(51) Int. Cl. A61K 31/55; A61P 25/22; A61P 25/00; A61P 25/18; A61P 25/24; A61P 25/08; A61P 29/00

(52) U.S. Cl. 514/217

(57) ABSTRACT

An oral suspension formulation comprising eslicarbazepine acetate and a pharmaceutically acceptable liquid vehicle.

### CROSS-REFERENCE TO RELATED APPLICATIONS

This application is a filing under 35 U.S.C. 371 of International Application No. PCT/PT2010/000038 filed Sep. 10, 2010, entitled “Oral Suspension Formulations of Esclicarbazepine Acetate,” which is a non-provisional of and claims priority to U.S. Provisional Patent Application No. 61/241,195 filed on Sep. 10, 2009, which applications are incorporated by reference herein in their entirety.

### FIELD OF INVENTION

This invention relates to formulations containing the active pharmaceutical ingredient (API) eslicarbazepine acetate, and to processes for making them. More particularly, the invention relates to oral suspension formulations containing eslicarbazepine acetate and to processes for making them.

### BACKGROUND

Eslicarbazepine acetate (ESL, S-(−)-10-acetoxy-10,11-dihydro-5H-dibenz/b,f/azepine-5-carboxamide) is a new voltage-gated sodium channel (VGSC) blocker that shares with carbamazepine (CBZ) the dibenzazepine nucleus bearing the 5-carboxamide substituent, but is structurally different at the 10,11-position (see BENES, J., PARADA, A., FIGUEIREDO, A. A., ALVES, P. C., FREITAS, A. P., LEARMONTH, D. A., CUNHA, R. A., GARRETT, J. & SOARES-DA-SILVA, P, (1999), “Anticonvulsant and sodium channel-blocking properties of novel 10,11-dihydro-5H-dibenz[b,f]azepine-5-carboxamide derivatives,” J. Med. Chem., 42, 2582-2587).

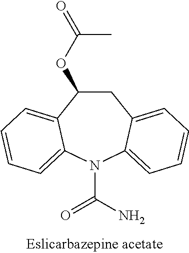 [SMILES: CC(=O)O[C@H]1Cc2ccccc2N(C(N)=O)c2ccccc21]

This molecular variation results in differences in metabolism, namely by preventing the formation of toxic epoxide metabolites, such as carbamazepine-10,11 epoxide, and unnecessary production of enantiomers or diastereoisomers of metabolites and conjugates (see HAINZL, D., PARADA, A. & SOARES-DA-SILVA, P. (2001), “Metabolism of two new antiepileptic drugs and their principal metabolites S(+)- and R(−)-10,11-dihydro-10-hydroxy carbamazepine”, Epilepsy Res, 44, 197-206), without losing pharmacological activity (see the above Benes reference).

ESL is useful as an anticonvulsant, for example in treating epilepsy, and also affective disorders, neuropathic pain and other pain disorders.

Preparation of pharmaceutical formulations for paediatric use raises additional concerns since tablets are often not suitable, being difficult to swallow and administer. A liquid formulation, for example a syrup or suspension, may be preferred. However, difficulties arise in maintaining chemical stability (e.g. limiting degradation of formulation components and production of impurities) and physical stability (e.g. maintaining viscosity, dissolution, aspect, pH, and preventing slow sedimentation and phase separation—caking) of such a liquid formulation. Moreover, ESL is a poorly water soluble drug, making it more complicated to formulate as a suspension formulation.

For example, there may be problems in controlling the chemical stability of the formulations. For example, eslicarbazepine acetate may degrade to form eslicarbazepine, R-licarbazepine and/or R-licarbazepine acetate. It is important to minimise the formation of such degradation products.

### OBJECTS OF THE INVENTION

It is an object of the invention to provide an oral suspension formulation containing eslicarbazepine acetate.

More particularly, it is an object of the invention to provide an oral suspension formulation containing eslicarbazepine acetate which has good physical and chemical stability.

### SUMMARY OF THE INVENTION

In accordance with one aspect of the present invention there is provided an oral suspension formulation comprising eslicarbazepine acetate and a pharmaceutically acceptable liquid vehicle.

The oral suspension formulation according to the invention is advantageously formulated with additional pharmaceutically acceptable excipients, as described in further detail below. In particular, the oral suspension formulation may further comprise a suspending agent and/or a wetting agent.

In accordance with another aspect of the present invention there is provided a process for making an oral suspension formulation comprising combining eslicarbazepine acetate with a pharmaceutically acceptable liquid vehicle.

The oral suspension formulation of the invention has good physical stability properties such as low levels of sedimentation (reduced or no caking) and easy re-dispersion on agitation. Additionally, the formulation may be used with a wide range of API particle sizes, for example from about 10 to about 70 μm. Moreover, the formulation exhibits good aspect, for example a low production of foam and homogeneity of suspension and low sediment compaction (low phase separation).

### DETAILED DESCRIPTION

The invention provides an oral suspension formulation comprising a therapeutically effective amount of eslicarbazepine acetate and a pharmaceutically acceptable liquid vehicle.

As used herein, and unless otherwise specified, a “therapeutically effective amount” of a compound is an amount sufficient to provide a therapeutic benefit in the treatment or management of a disease or disorder, or to prevent, delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” encompasses an amount that improves overall therapy, reduces, prevents or avoids symptoms or causes of disease or disorder, or enhances the therapeutic efficacy of another therapeutic agent.

The active ingredient, eslicarbazepine acetate, is present in an amount of from about 1 to about 10 w/v % of the suspension formulation, preferably from about 3 to about 7 w/v % of the formulation, more preferably from about 4 to about 6 w/v % of the formulation and most preferably about 5 w/v % of the formulation.

Preferably, the liquid vehicle is present in the suspension formulation in an amount of from about 85 to about 95 w/v %, more preferably from about 90 to about 95 w/v % of the suspension formulation.

The liquid vehicle may comprise an aqueous-based medium, for example, water, propylene glycol, aqueous sorbitol solution, aqueous buffer solution as described herein, or a mixture of two or more of these.

Therefore in a preferred embodiment, the liquid vehicle includes a buffer solution having a pH in the range of about 6.8 to about 7.0, preferably about 6.9. Preferably, the buffer is a phosphate buffer, more preferably with a high buffering capacity, for example the pH of the formulation containing such buffer solution varies within no more than 3 pH units the above-noted range, preferably no more than 2 pH units the above-noted range in the final oral suspension formulation. Desirably, the buffer solution is present in an amount of from about 50 to about 90 vol % of the liquid vehicle, i.e. from about 45 to about 85 w/v % of the oral suspension formulation. Optionally, the buffer solution is present in an amount of from about 50 to about 90 vol % of the formulation. In an embodiment, the buffer is formed using potassium di-hydrogen phosphate and water with sodium hydroxide, or with sodium hydroxide and aqueous hydrochloric acid (HCl (aq)). However, alternatively the buffer is formed using potassium di-hydrogen phosphate with disodium hydrogen phosphate dihydrate. In each case, an appropriate amount of water is added, and the final pH can be set using sodium hydroxide and or HCl (aq), as necessary. Examples of specific buffers are as follows:

(1) The buffer was formed by the following process: about 3.5 g of potassium di-hydrogen phosphate was transferred into a beaker of 1000 mL and dissolved with 800 mL of water; 1 g of solid sodium hydroxide and 1.43 g of HCl (conc.) were added. The volume was made up to 1000 mL with water. The pH was set to 6.9±0.05 with sodium hydroxide solution at 1 mol/L or aqueous HCl solution at 1 mol/L such that [Phosphate]=0.026 M and [NaCl]=0.091 M.

(2) The buffer was formed by the following process: about 6.80 g of potassium di-hydrogen phosphate was transferred into a beaker of 1000 mL and dissolved with 800 mL of water; 33 mL of sodium hydroxide solution at 1 mol/L was added and the volume made up to 1000 mL with water. The pH was set to 6.90±0.05 with sodium hydroxide solution at 1 mol/L such that [Phosphate]=0.050 M and [NaCl]=0.088 M.

(3) The buffer was formed by the following process: acid/base conjugate buffer—about 3.4 g of potassium di-hydrogen phosphate was transferred into a beaker of 1000 mL and dissolved with 800 mL of water; 4.45 g of di-sodium hydrogen phosphate di-hydrate was added and the volume made up to 1000 mL with water. The pH was set to 6.90±0.05 with sodium hydroxide solution at 1 mol/L or aqueous HCl solution at 1 mol/L such that [Phosphate]=0.056 M and [NaCl]=0.087 M.

(4) The buffer was formed by the following process: about 6.8 g of potassium di-hydrogen phosphate was transferred into a beaker of 1000 mL and dissolved with 800 mL of water; 8.9 g of di-sodium hydrogen phosphate di-hydrate was added and the volume made up to 1000 mL with water. The pH was set to 6.90±0.05 with sodium hydroxide solution at 1 mol/L or aqueous HCl solution at 1 mol/L such that [Phosphate]=0.113 M and [NaCl]=0.176 M.

It was discovered that when the liquid vehicle comprises an aqueous solution of sorbitol, the presence of aqueous sorbitol solution above about 50 vol % of the liquid vehicle resulted in a significant reduction in the dissolution properties of the suspension, whereas the presence of aqueous sorbitol solution below about 10 vol % of the liquid vehicle resulted in significant phase separation of the oral suspension formulation. Preferably, therefore, the aqueous sorbitol solution is present in an amount of from about 10 to about 50 vol % of the liquid vehicle, i.e. from about 9 to about 45 w/v % of the oral suspension formulation. Optionally, the aqueous sorbitol solution is present in an amount of from about 10 to about 50 vol % of the formulation.

It was also found that the use of an aqueous sorbitol solution in the liquid vehicle enhances the stability, particularly the physical stability (e.g. reduced phase separation and increased ease of re-dispersibility) across a wide range of ESL particle sizes, of the formulation, and that there is a synergistic enhancement in stability when the aqueous sorbitol solution is used in combination with xanthan gum as a suspending agent.

Therefore in certain preferred embodiments, the liquid vehicle may comprise an aqueous solution of sorbitol. When the aqueous sorbitol solution is present, the sorbitol is preferably used in an aqueous solution in an amount from about 50 to about 90 w/v %. Preferably, sorbitol comprises about 70% w/v % (i.e., from about 68.5 to about 71.5 w/v % in from about 28.5 to about 31.5 parts water, preferably approximately 70 parts sorbitol in approximately 30 parts water).

As noted above, it was found that a combination of xanthan gum as suspending agent and aqueous sorbitol solution in the liquid vehicle is particularly effective and leads to a more stable formulation than other combinations of suspending agent and liquid vehicle. It was also found that this combination of xanthan gum and aqueous sorbitol solution provides the desired viscosity to the oral suspension, the desired viscosity being defined elsewhere herein.

In particular, this combination provides a formulation with good viscosity, low sedimentation, and the ability to form acceptable formulations over a wide range of agitation times, including from about 30 minutes to about 2 hours.

Therefore in a preferred embodiment the oral suspension formulation further comprises a suspending agent. The suspending agent may be selected from selected from acacia gum, alginic acid, carbomer, carboxymethylcellulose sodium, ceratonia, cottonseed oil, dextrin, dextrose, gelatin, guar gum, hydrogenated vegetable oil type I, hydroxyethyl cellulose, hydroxyethylmethyl cellulose, hydroxypropyl cellulose, low substituted hydroxypropyl cellulose, hypromellose, povidone, magnesium aluminium silicate, maltodextrin, maltose, methylcellulose, ethylcellulose, microcrystalline cellulose, polydextrose, polyethylene oxide, polymethacrylates, sodium alginate, starch, pregelatinised starch, stearic acid, xanthan gum, sucrose and zein. In certain embodiments, the suspending agent may be microcrystalline cellulose. In certain embodiments, the suspending agent may be a combination of microcrystalline cellulose and carboxymethyl cellulose sodium (e.g., Avicel™ RC-591).

In certain embodiments, xanthan gum may be particularly effective as the suspending agent. Moreover, it was discovered that xanthan gum provided good viscosity in the oral suspension formulation. For example, it is desirable that the formulation according to the invention should have a viscosity in the range of from about 120 to about 450 cP, or in the range of from about 150 to about 300 cP, or in the range of from about 180 cP to about 400 cP, or in the range of from about 200 cP to about 380 cP, or in the range of from about 250 cP to about 350 cP. It has been found that xanthan gum provides a viscosity in this range over a wide range of concentrations of xanthan gum. Thus, when xanthan gum is used, the viscosity is not very sensitive to changes in the concentration of the gum. It is preferred that, the suspending agent is present in an amount from about 0.1 to about 0.5 w/v % of the suspension formulation, i.e., from about 0.1 mg to about 0.5 mg per 100 mL of suspension, such as 0.1, 0.2, 0.3, 0.4 or 0.5 w/v %, most preferably from about 0.2 to about 0.3 w/v % of the suspension formulation.

In a preferred embodiment, the formulation further comprises a wetting agent. Suitable wetting agents include for example gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters or polysorbates (e.g., TWEEN™), polyethylene glycols, polyoxyethylene stearates, phosphates, sodium lauryl sulphate, poloxamer, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, non-crystalline cellulose, magnesium aluminum silicate, triethanolamine, polyvinyl alcohol, polyvinylpyrrolidone (or PVP), tyloxapol (also known as superinone or triton), and combinations thereof. The wetting agent may be a polyoxyethylene sorbitan fatty acid ester, such as polysorbate 80. Alternatively, the wetting agent may be a polyoxyethylene stearate (also known as poly(ethylene glycol) stearate) as the wetting agent, especially polyoxy 100 stearate. Myrj™ 59P, also known as Myrj™ S100, (polyethylene glycol 100 stearate) was found to be particularly suitable as the wetting agent. It was also discovered that a much lower amount of wetting agent could be used as compared to the amounts generally used in such oral suspension formulations. For example, the guidance in the field (Handbook of Pharmaceutical Excipients, 4^{th} edition, American Pharmaceutical Association, 2003) states that the wetting agent should be used in an amount of 0.5 to 5 w/v %, whereas oral suspension formulations of the present invention comprise a wetting agent in the range of from about 0.1 to about 0.5 w/v % of the suspension formulation. Desirably, the wetting agent is present in an amount of from about 0.05 to about 5 w/v %, most preferably from about 0.1 to about 0.5 w/v % of the formulation, such as 0.1, 0.2, 0.3, 0.4, 0.5 w/v % of the formulation.

The use of a polyoxyethylene stearate, particularly polyoxy 100 stearate, especially Myrj™ 59P, improves wettability of the eslicarbazepine acetate as well as providing improved homogeneity of eslicarbazepine acetate particles in the formulation. It may also show some synergy with the suspending agent, e.g., xanthan gum, in improving these two characteristics.

In a preferred embodiment, the formulation further comprises an antimicrobial agent. Suitable antimicrobial agents include Sorbic acid, Sodium sorbate, Potassium sorbate, Calcium sorbate, Benzoic acid, Sodium benzoate, Potassium benzoate, Calcium benzoate, Ethyl para-hydroxybenzoate, Sodium ethyl para-hydroxybenzoate, Propylparaben, Propyl para-hydroxybenzoate, Sodium propyl para-hydroxybenzoate, Methylparaben, Methyl para-hydroxybenzoate, Sodium methyl p-hydroxybenzoate, Sulphur dioxide, Sodium sulphite, Sodium bisulphite, Sodium hydrogen sulphite, Sodium metabisulphite, Potassium metabisulphite, Potassium sulphite, Calcium sulphite, Calcium hydrogen sulphite, Potassium bisulphite, Potassium hydrogen sulphite, Biphenyl, Diphenyl, Orthophenyl phenol, Sodium orthophenyl phenol, Thiabendazole, Nisin, Natamycin, Pimaracin, Formic acid, Sodium formate, Calcium formate, Hexamethylene tetramine, Hexamine, Formaldehyde, Dimethyl dicarbonate, Potassium nitrite, Sodium nitrite, Sodium nitrate, saltpetre, Potassium nitrate, Acetic acid, Potassium acetate, Sodium acetate and ahydrous, Sodium diacetate, Calcium acetate, Ammonium acetate, Lactic acid, Propionic acid, Sodium propionate, Calcium propionate, Potassium propionate, Boric acid, Sodium tetraborate, methylparaben, propylparaben, or a combination thereof. Preferably, the antimicrobial agent is a combination of methylparaben and propylparaben. Desirably, the antimicrobial agent is present in a total amount of from about 0.1 to about 0.5 w/v %, such as 0.1, 0.2, 0.3, 0.4, or 0.5 w/v %, most preferably from about 0.15 to about 0.25 w/v %, of the suspension formulation.

In certain embodiments, the formulation further comprises other pharmaceutically acceptable excipients, such as one or more sweetening agents, and/or one or more flavouring agents.

Suitable sweetening agents are well known to the skilled person and are selected from gluconate, aspartame, cyclamate, saccharin sodium, xylitol and maltitol, or mixtures thereof.

In the present formulation, the sweetening agent is suitably saccharin sodium. When present, the sweetening agent is preferably provided in an amount of from about 0.05 to about 0.15 w/v % of the suspension formulation, such as 0.05, 0.075, 0.1 or 0.15 w/v % of the suspension formulation.

Suitable flavouring agents are well known to the skilled person and are selected from chocolate, bubble gum, cocoa, coffee, fruit flavouring (such as wild cherry, banana, grape, peach, and, raspberry), oil of peppermint, oil of spearmint, oil of orange, mint flavour, anise flavour, honey flavour, vanilla flavour, tea flavour and verbena flavour, and various fruit acids such as citric acid, ascorbic acid and tartaric acid, or mixtures thereof. In the present formulation, the flavouring agent is selected from golden syrup flavour, raspberry flavour, caramel flavour, bubble gum flavour, and the like, including combinations thereof. When present, the flavouring agent is preferably provided in an amount of from about 0.05 to about 5 w/v % of the suspension formulation, such as 0.1, 0.2, 0.3, 0.4, 0.5, 0.6, 0.7, 0.8, 0.9, 1, 1.5, 2, 2.5, 3, 3.5, 4, 4.5, or 5 w/v % of the suspension formulation.

Thus, a preferred oral dosage formulation according to the invention comprises:

Eslicarbazepine acetate—from about 4 to about 6 w/v % of the formulation

Xanthan gum—from about 0.2 to about 0.3 w/v % of the formulation

Polyoxy 100 stearate—from about 0.2 to about 0.3 w/v % of the formulation

Liquid Vehicle—from about 85 to 95 w/v % of the formulation

Buffer solution—from about 50 to about 90 vol % of the liquid vehicle

Aqueous sorbitol solution—from about 10 to about 50 vol % of the liquid vehicle.

More preferably, this formulation further comprises one or more of the following:

Methylparaben & propylparaben—from about 0.1 to about 0.3 w/v % of the formulation

Sweetener—from about 0.05 to about 0.15 w/v % of the formulation

Flavouring agent—from about 0.05 to about 5 w/v % of the formulation.

The aqueous sorbitol solution in this formulation preferably comprises from about 60 to about 80 w/v % sorbitol, more preferably from about 65 to about 75 w/v % sorbitol and most preferably about 70 w/v % sorbitol.

The formulation can be provided in any desired quantity typically 50 mL, 100 mL, 150 mL or 200 mL. Generally the formulation is provided in a bottle of a size appropriate to the desired quantity.

Typically, the formulation will be administered to deliver from 10 mg/kg/day up to 30 mg/kg/day of the active ingredient, eslicarbazepine acetate.

Typically, the formulation will be administered to deliver up to 1200 mg/day, or up to 1800 mg/day, such as 200, 400, 600, 800, 1000, 1200, 1400, 1600 or 1800 mg/day of the active ingredient. In certain embodiments, the formulation will be administered to deliver 200, 400, 600, 800, 1000, 1200, or 1800 mg/day of the active ingredient.

The formulation may be administered to a patient in need thereof by measuring a therapeutically effective quantity of the formulation and administering it orally to the patient. A typical therapeutically effective dosage would be from about 4 to about 40 mL, for example from about 8 to about 20 mL of the formulation. In certain embodiments, a typical therapeutically effective dosage would be from about 8 to about 20 mL of the formulation. The formulation can advantageously be administered once-daily, as further described in WO2006/121363, which is incorporated herein by reference.

The formulation according to the invention is particularly preferred for paediatric use. The formulation is particularly preferred for use in treating epilepsy, neuropathic pain and other pain conditions such as migraine and fibromyalgia. It can also be used in the treatment of other disorders, such as affective disorders, schizoaffective disorders, bipolar disorders, attention disorders, anxiety disorders, neuropathic pain-related disorders, sensorimotor disorders, vestibular disorders, or nervous function alterations in degenerative and post-ischemic diseases.

Examples of affective disorders include depression, pre-menstrual dysphoric disorder, post-partum depression, post-menopausal depression, anorexia nervosa, bulimia nervosa, and neurodegeneration-related depressive symptoms.

Examples of schizoaffective disorders include schizodepressive syndromes, schizophrenia, extreme psychotic states, schizomanic syndromes, dysphoric and aggressive behaviour, episodic dyscontrol or intermittent explosive disorder, and borderline personality disorder.

Examples of bipolar disorders include unstable bipolar disorder with rapid fluctuations (rapid cyclers), manic-depressive disorders, acute mania, mood episodes, and manic and hypomanic episodes.

Examples of attention disorders include attention deficit hyperactivity disorders and other attention disorders, such as, for example, autism.

Anxiety disorders include conditions such as, for example, social anxiety disorders, post traumatic stress disorder, panic, obsessive-compulsive disorder, alcoholism, drug withdrawal syndromes, and cravings.

The neuropathic pain, neuropathic pain-related disorders and pain conditions that may be treated according to the methods of the present disclosure include, by way of example, neuropathic pain and associated hyperalgesia, including trigeminal, herpetic, post-herpetic and tabetic neuralgia, diabetic neuropathic pain, migraines, tension-type headaches, causalgia, fibromyalgia and deafferentation syndromes such as, for example, brachial plexus avulsion. In certain embodiments the neuropathic pain and associated hyperalgesia is selected from neuropathic pain and associated hyperalgesia, including trigeminal, herpetic, post-herpetic and tabetic neuralgia, diabetic neuropathic pain, migraines, tension-type headaches, causalgia, and deafferentation syndromes such as, for example, brachial plexus avulsion.

Examples of sensorimotor disorders include restless legs syndrome, spasticity, hemifacial spasm, nocturnal paroxysmal dystonia, brain ischemia associated motor and sensitive deficits, Parkinson's disease and Parkinsonian disorders, antipsychotic-induced motor deficits, tardive dyskinesia, episodic nocturnal wandering, and myotonia.

Exemplary vestibular disorders include tinnitus or other inner ear/cochlear excitability related diseases, such as, for example, neuronal loss, hearing loss, sudden deafness, vertigo, and Meniere's disease.

One skilled in the art will understand that these conditions are exemplary only, and will understand from the disclosure what other diseases and conditions would be considered to be within the scope of the present disclosure.

According to another aspect of the invention there is provided a process for making an oral suspension formulation as defined above, comprising (1) preparing an aqueous buffer solution, preferably having a pH of about 6.9; (2) adding a suspending agent to the buffer solution; (3) adding a wetting agent to the buffer solution; (4) adding eslicarbazepine acetate to the buffer solution; (5) and adding the aqueous sorbitol solution to the buffer solution. Preferably, steps (1) to (5) are carried out in the above sequence so as to maintain satisfactory properties such as viscosity, if necessary stifling the formulation during and/or after adding the materials.

Preferably, the buffer solution is as described herein. Preferably step (1) is performed as described herein. Preferably the suspending agent, wetting agent, sorbitol solution, anti-microbial agent, sweetening agent and/or flavouring agent are as described herein.

If present, the antimicrobial agent, the sweetening agent and/or the flavouring agent can also be added, preferably between steps (4) and (5), or during step (4). In one embodiment the antimicrobial agent is added during step (4) and the sweetening agent and/or flavouring agent is/are added between step (4) and step (5).

It was noted that, when the wetting agent and the suspending agent are added to the buffer in step (2), the amount of other formulation components (assay) was affected, for example the antimicrobial agent assay was reduced. Adding the suspending agent separately from and in advance of the wetting agent avoided this problem.

It was also found to be advantageous to add the antimicrobial agent after the other excipients, as to add it straight after step (1) affected the pH stability of the formulation. A similar effect was seen with the addition of the ESL when added straight after step (1), and additionally the ESL assay decreased when this component was added straight after step (1).

### EXAMPLES

Certain embodiments are exemplified in the following non-limiting example. It will be apparent to those skilled in the art that many modifications, both to materials and methods, can be practised without departing from the spirit and scope of this disclosure.

### Example 1

The following exemplary composition was prepared by the process described below.

| | Quantity | | |
|---|---|---|---|
| | (mg/mL unless | | Reference |
| Start-material | stated otherwise) | Function | standard |
| ESL | 40-60 | Active substance | Monograph |
| Xanthan gum | 2-3 | Suspending agent | Ph. Eur. |
| Myrj ™ 59P | 2-3 | Solubilising agent | Ph. Eur. |
| Methylparaben & | 1-3 | Antimicrobial agent | Ph. Eur. |
| propylparaben | | | |
| Saccharin sodium | 0.5-1.5 | Sweetening agent | Ph. Eur. |
| Flavouring | 0.5-50 | Flavouring agent | Monograph |
| Buffer pH 6.9 | 0.7 mL | Liquid Vehicle | Monograph |
| Sorbitol 70% | to 1 mL | Liquid Vehicle | Ph. Eur. |

The composition was made as follows:
The buffer solution (pH 6.9) was prepared as follows:
about 3.4 g of potassium di-hydrogen phosphate was transferred into a beaker of 1000 mL and dissolved with 800 mL of water; 4.45 g of di-sodium hydrogen phosphate di-hydrate was added and the volume was made up to 1000 mL with water. The pH was set to 6.9±0.05 with sodium hydroxide solution at 1 mol/L or aqueous HCl solution at 1 mol/L such that [Phosphate]=0.056 M and [NaCl]=0.087 M.
Xanthan gum was added to the buffer solution pH 6.9 and allowed to macerate for 1 hour, under stirring (IKA™ position 6*).
Polyoxy 100 stearate (Myrj™ 59P) was then added to the mixture and allowed to stir for 30 minutes (IKA™ position 6*).
ESL was added to the suspension, under stirring, followed by addition of the methylparaben and the propylparaben before stirring for 1 hour (IKA™ position 6*).
The saccharin sodium and the flavouring were added, and the volume completed with sorbitol 70%, slowly added under stifling. The suspension was allowed to stir for 1 hour (IKA™ position 6*).
Bottles were filled with 200 mL of the resultant suspension.
* Refers to the preferred setting using an IKA™ stirrer.

### Example 2

The following exemplary composition was also prepared by the process described above:

| | Quantity | | |
|---|---|---|---|
| | (mg/mL unless | | Reference |
| Start-material | stated otherwise) | Function | standard |
| ESL | 50 | Active substance | Monograph |
| Xanthan gum | 2.5 | Suspending agent | Ph. Eur. |
| Myrj ™ 59P | 2.5 | Solubilising agent | Ph. Eur. |
| Methylparaben & | 2 | Antimicrobial agent | Ph. Eur. |
| propylparaben | | | |
| Saccharin sodium | 1.0 | Sweetening agent | Ph. Eur. |
| Bubble gum | 1.0 | Flavouring agent | Monograph |
| Buffer pH 6.9 | 0.7 mL | Liquid Vehicle | Monograph |
| Sorbitol 70% | to 1 mL | Liquid Vehicle | Ph. Eur. |

It will be appreciated that the invention described above may be modified within the scope of the claims.

1. An oral suspension formulation comprising a therapeutically effective amount of eslicarbazepine acetate and a pharmaceutically acceptable liquid vehicle.

2. The formulation as defined in claim 1, further comprising a suspending agent.

3. The formulation as defined in claim 2, wherein the suspending agent is xanthan gum.

4. The formulation as defined in claim 2, wherein the suspending agent is present in an amount from 0.1 to 0.5 w/v % of the formulation.

5. The formulation as defined in claim 1, further comprising a wetting agent.

6. The formulation as defined in claim 5, wherein the wetting agent is a polyoxyethylene stearate, such as polyoxy 100 stearate.

7. (canceled)

8. The formulation as defined in claim 5, wherein the wetting agent is present in an amount 0.05 to 5 w/v % of the formulation.

9. The formulation as defined in an claim 1, further comprising an antimicrobial agent.

10. The formulation as defined in claim 9, wherein the antimicrobial agent comprises methylparaben and/or propylparaben.

11. (canceled)

12. The formulation as defined in claim 1, wherein the liquid vehicle is present in an amount from 85 to 95 w/v % of the formulation.

13. The formulation as defined in claim 1, wherein the liquid vehicle comprises a buffer solution having a pH in a range of 6.8 to 7.0.

14. The formulation as defined in claim 13, wherein the buffer solution is present in an amount from 50 to 90 vol % of the liquid vehicle.

15. The formulation as defined in claim 1, wherein the liquid vehicle comprises an aqueous sorbitol solution, that is present in an amount from 10 to 50 vol % of the liquid vehicle.

16. (canceled)

17. An oral dosage formulation comprising:
Eslicarbazepine acetate—40-60 mg/mL of the formulation;
Xanthan gum—2-3 mg/mL of the formulation;
Polyoxy 100 stearate—2-3 mg/mL of the formulation;
Buffer solution—0.7 mL; and
Aqueous sorbitol solution—to 1 mL, wherein the sorbitol solution comprises 65 to 75 w/v % sorbitol.

18. (canceled)

19. A method comprising utilizing sorbitol solution in combination with xanthan gum in an oral suspension formulation comprising eslicarbazepine acetate to improve the physical stability thereof.

20. The method as defined in claim 19, comprising utilizing from 9 to 45 w/v % aqueous sorbitol solution based on the volume of the formulation, and 0.2 to 0.3 w/v % xanthan gum based on the volume of the formulation.

21. A process for making an oral suspension formulation comprising eslicarbazepine acetate, comprising (1) preparing a buffer solution; (2) adding a suspending agent, such as xanthan gum to the buffer solution; (3) adding a wetting agent to the buffer solution; (4) adding eslicarbazepine acetate to the buffer solution; (5) and adding aqueous sorbitol solution to the buffer solution.

22. (canceled)

23. The process as defined in claim 21, wherein the suspending agent is added in an amount of from 0.1 to 0.5 w/v % of the formulation.

24. The process as defined in claim 21, wherein the wetting agent is polyoxy 100 stearate, and is optionally added in an amount of from 0.05 to 5 w/v % of the final formulation.

25. (canceled)

26. The process as defined in claim 21, wherein the buffer solution has a pH in a range of from 6.8 to 7.0.

27. The process as defined in claim 21, wherein the buffer solution prepared makes up from 45 to 85 w/v % of the final formulation.

28. (canceled)

29. The process as defined in claim 21, wherein an antimicrobial agent is added during step (4).

30. The process as defined in claim 29, wherein the antimicrobial agent comprises methylparaben and/or propylparaben and is optionally added in an amount of from 0.1 to 0.5 w/v % of the final formulation.

31. (canceled)

32. The process as defined in claim 21, wherein a flavouring agent is added between step (4) and step (5).

33. The process as defined in claim 21, wherein a sweetening agent is added between step (4) and step (5).

34. (canceled)

35. The formulation as defined in claim 10, wherein the antimicrobial agent is methylparaben.

36. The formulation as defined in claim 1, wherein the liquid vehicle is water.
